(19) United States
(12) Patent Application Publication
(10) Pub. No.: US 2005/0033185 A1
(43) Pub. Date: Feb. 10, 2005

(54) Method for correcting vessel and background light intensities used in beer's law for light scattering in tissue

(75) Inventor: Robert M. Danen, San Jose, CA (US)

Correspondence Address: CAESAR, RIVISE, BERNSTEIN,;COHEN & POKOTILOW, LTD., 11TH FLOOR, SEVEN PENN CENTER, 1635 MARKET STREET, PHILADELPHIA, PA, 19103-2212, US

(73) Assignee: Cytometrics, LLC, Exton, PA (US)

(21) Appl. No.: 10/635,353

(22) Filed: Aug. 6, 2003

(51) Int. Cl. A61B 5/00

(52) U.S. Cl. 600/476

(57) ABSTRACT

A method to correct the intensities of an absorption image for light scattered within foreground tissue. Specifically the amount of light scattered between the focal plane of the probe and the tissue surface is estimated from the image. This scattered light contribution is then subtracted from the original image.

### FIELD OF THE INVENTION

The invention pertains to methods for intravital microscopy and more particularly to a method for correcting the vessel and background intensities used in Beer's Law for light scattering in tissue from an orthogonal polarized spectral imaging system.

### BACKGROUND OF INVENTION

A recent innovation in the field of intravital microscopy is the introduction of a non-invasive instrument that produces high-contrast microvascular images which can be used in surgery, research and diagnostic applications. In particular, orthogonal polarization spectral (OPS) imaging systems (e.g., the device sold under the tradename CYTOSCAN® by Cytometrics LLC of Exton, Pa.) utilize non-invasive instruments that produce an image by using scattered polarized light which creates a “virtual” light source within the observed tissue. The images generated by the OPS imaging systems allow doctors/clinicians the ability to visualize and measure real time images of the living being's microcirculation without the need for fluorescent dyes or transillumination.

However, as will be discussed in detail later, the photons from the light transmitted from the OPS imaging systems to the underlying tissue structure, travel in all directions. These photons scatter in all directions within the tissue, thereby distorting the relative intensities between blood vessel regions and background regions.

The following discussion relates to current references or activities associated with microvessel imaging.

“Modeling and simulation of illumination effects for evaluation of microvessels of the conjunctiva” (Wick, Loew, and Kurantsin-Mills 1996, Modeling in Physiology, H1229-H1239) involves modeling the reflection of source light within the conjunctiva.

To the best of Applicant's knowledge, researchers (Chance, et al., Yodh, et al.) at the University of Pennsylvania have developed techniques that measure oxygenation of hemoglobin. Jacques (Univ. of Oreg.) has developed a polarization method to study the structure of epidermal tissue. However, the techniques referred to by Chance, et al., Yodh, et al. and Jacques measure optical properties of blood and tissues generally at near-infrared wavelengths. Most attempts to reconstruct both the absorption and scattering of tissue are on macroscopic scales (˜0.5 mm). At near-infrared wavelengths, light scattering dominates absorption; thus, elaborate inversion techniques are needed to determine the tissue optical parameters.

Thus, there remains a need for a method that solves for light scattering as a perturbative method in order to correct an absorption image when tissue vessels reside in the focal plane of an orthogonal polarization spectral (OPS) imaging system.

### SUMMARY OF THE INVENTION

A method for correcting light intensities from blood vessels and background tissue beneath tissue surface in a living being that are exposed to polarized light from an orthogonal polarized spectral (OPS) imaging system. The method comprises the steps of: emitting polarized light at tissue comprising blood vessels and background tissue, wherein the blood vessels are located at a focal plane of the OPS imaging system and wherein a foreground region is formed between the focal plane and the tissue surface; collecting de-polarized light that has impacted the blood vessels and that has experienced scattering within the foreground region and wherein the de-polarized light emerges from the tissue surface; generating a first image based on the collected de-polarized light; estimating the intensity of light scattered in the foreground region; and subtracting the intensity of light scattered in the foreground region from the first image to generate a corrected image based on focal plane light intensities.

### DESCRIPTION OF THE DRAWINGS

FIG. 1 is a block diagram of an exemplary apparatus that uses orthogonal polarization spectral (OPS) imaging and depicts how polarized light, emitted from the OPS probe at target tissue, interacts with underlying tissue structure and is captured by the probe for processing;

FIG. 2 is a physical model of light propagation within tissue that originated from an orthogonal polarization spectral (OPS) imaging system;

FIG. 3 is a flow diagram of the method of the present invention; and

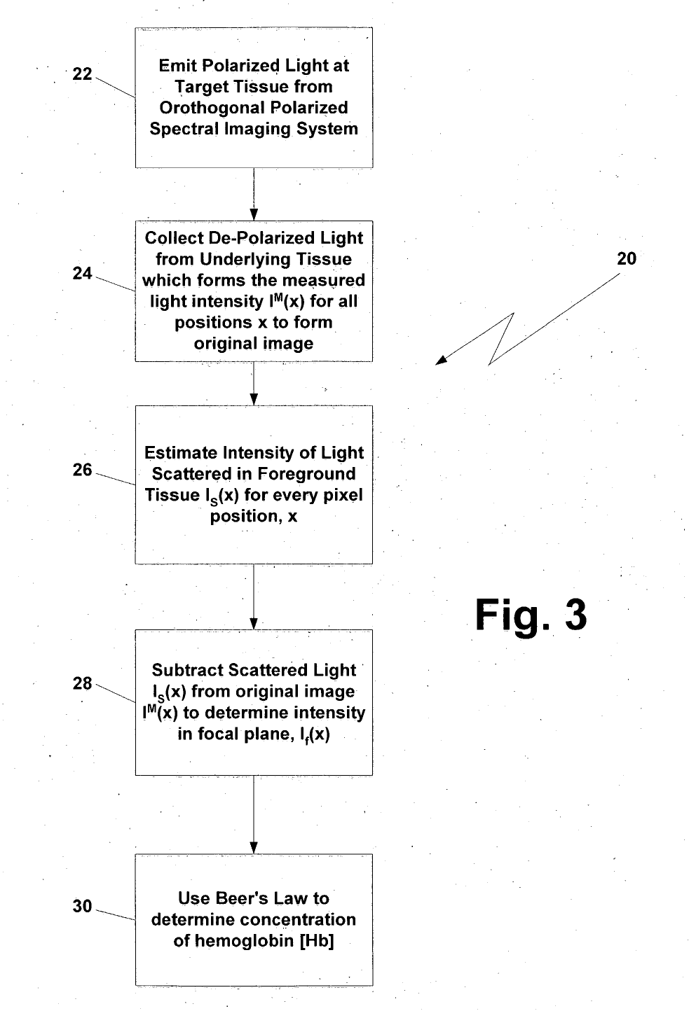

FIG. 4 is a flow diagram of the software of the present invention.

### DETAILED DESCRIPTION OF THE PREFERRED EMBODIMENTS

The invention is a method to correct the intensities of an absorption image for light scattered within foreground tissue using orthogonal polarization spectral (OPS) imaging. Specifically, the amount of light scattered between the focal plane of the probe and the tissue surface is estimated from the image. This scattered light contribution is then subtracted from the original image.

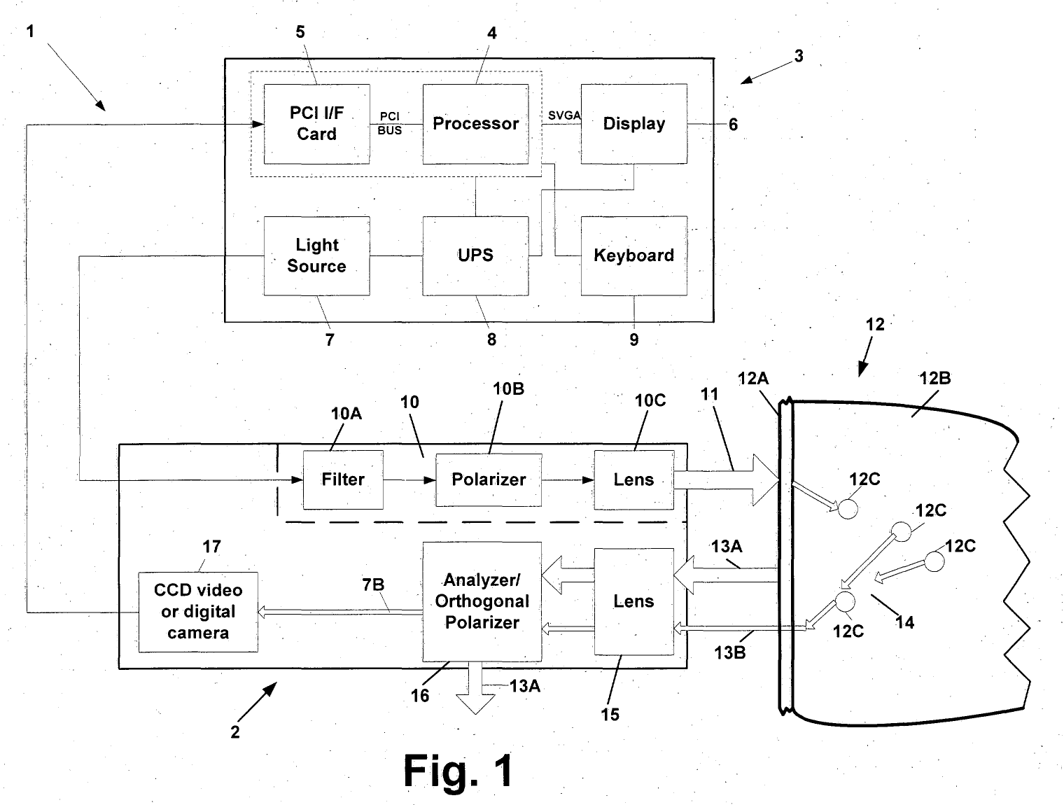

Before the method of the present invention is discussed, a summary of an exemplary device using OPS is described, such as the device marketed under the tradename CYTOSCAN® by Cytometrics LLC of Exton, Pa. As shown in FIG. 1, the device 1 comprises a probe 2 and a base unit 3. The base unit 3 comprises, among other things, a processor 4 (e.g., Pentium II processor), a PCI interface card 5, a display 6, a light source 7 (e.g., Welch-Allyn metal-halide arc lamp), an uninterruptible power supply (UPS) 8, and a keyboard 9. The probe 2 is electrically and optically coupled to the base unit 3 through a harness (not shown).

The probe 2 comprises a lens assembly 10 that emits a beam of polarized light 11 filtered by filter 10A at 550 nm (the importance of which is discussed below) aimed at the target tissue 12. The majority (e.g., 90%) of the polarized light 11 is reflected from the target tissue surface 12A as glare 13A while the remaining portion of the polarized light penetrates deeply through the skin and impacts underlying tissue 12B, causing multiple scattering events 14. When this polarized light of 550 nm impacts various structures, it back-lights the structures, causes hemoglobin 12C to fluoresce (550 nm is the isobestic point for hemoglobin) and de-polarizes/scatters the light. This de-polarized light 13B is reflected back to and out of the target tissue surface 12A. As a result, the probe 2 receives both the glare 13A and the de-polarized light 13B which are passed through a lens 15 and an analyzer/orthogonal polarizer (APO) 16 inside the probe 2. The APO 16 (which is orthogonal to the first polarizer 10B) filters out the glare 13A and passes the de-polarized light 13B to a CCD video (or digital) camera 17 which converts the optical signal to an electrical (e.g., digital) signal for processing by the processor 4 and ultimately for display on the display 6 where an image (e.g., red blood cells, vessels, etc., ) is formed of the structures impacted by the incoming light and the scattered light.

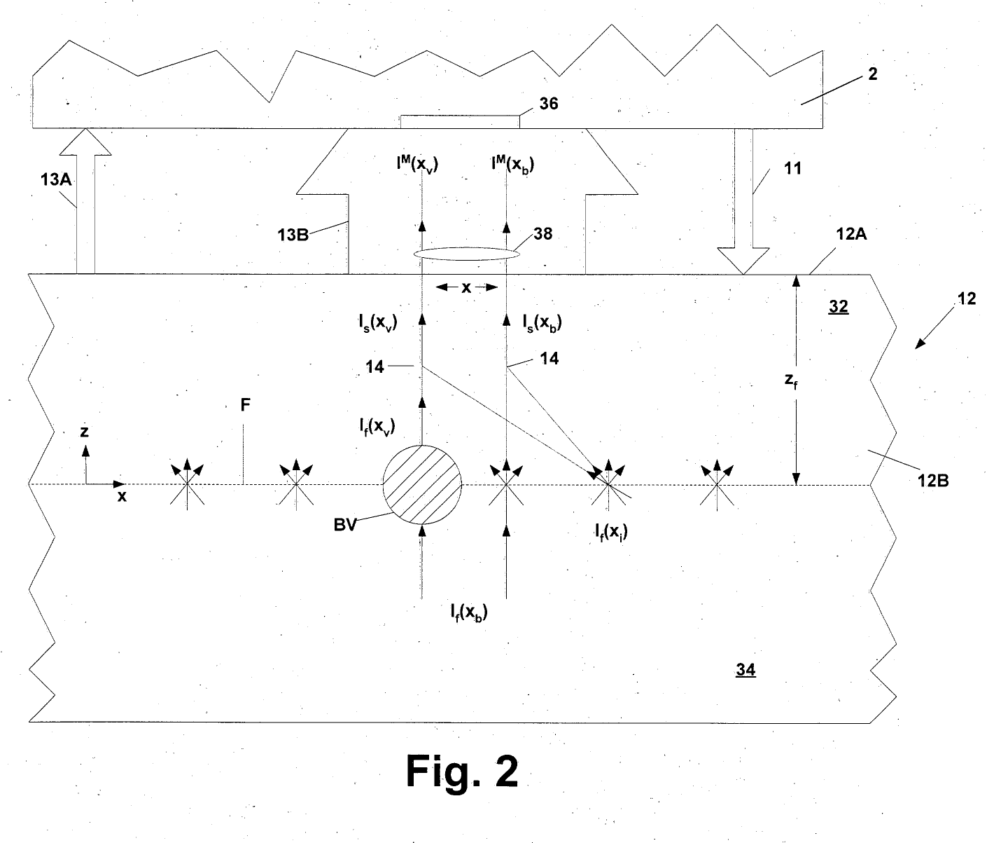

When the tissue vessels (e.g., blood vessels, BV) are positioned in the focal plane F (FIG. 2) of the OPS probe 2, an incorrect estimation of the hemoglobin (Hb) occurs. In particular, light scatters in foreground tissue 32, the region of tissue between the tissue surface 12A and focal plane F of the OPS imaging system; a diffuse background 34 region is also shown in FIG. 2. Because the light field is diffuse in the focal plane F, photons travel in all directions, not just the direction defined by the collection optics (e.g., lens 15/AOP 16) of the probe 2. These photons can scatter within the tissue 12B, distorting the relative intensities between vessel and background regions. This scattered light distorts the contrast of objects in the focal plane F. Because the concentration of Hb [Hb] is determined by measuring the quantity of light absorbed by vessels (e.g., blood vessels BV) in the focal plane F, light scattering in-the foreground tissue 32 degrades measurements of [Hb].

Generally, by measuring the intensity of light absorbed by a blood vessel BV, [Hb] can be determined using Beer's law (A=2-log₁₀% T, where A is absorbance and T is transmittance). However, FIG. 2 shows a diagram of light propagation within surface tissue for the OPS imaging system. As a result of the design. of the OPS imaging system, all detected light travels from below the focal plane before reaching the tissue surface and the light field is diffuse within the focal plane F. The intensity of light measured by the OPS probe 2 at position x on the tissue surface (I^{M}(x)) is the sum of two components: light traveling directly from position x on the focal plane (I_{f}(x)) and light scattered from other positions on the focal plane F, (Iₛ(x)):
I^{M}(x)=I_{f}(x)e^{-μ}^{_{f}}^{z}^{_{f}}+Iₛ(x), where
I^{M}(x)=light intensity measured at position x on the tissue surface 12A;
I_{f}(x)=light intensity that travels without scattering from position x on the focal plane (F) to position x on the tissue surface 12A;
Iₛ(x)=light intensity that scatters at 14 from other positions xᵢ(xᵢ≠x) on the focal plane (F) to position x on the tissue surface 12A.
   In the above equation, μₜ, is the transport coefficient of the scattering medium and z_{f} is the distance between the focal plane F and tissue surface 12A.

As mentioned previously, use of measured intensities I^{M} as representative of the light distribution in the focal plane F where vessels reside, yields an incorrect estimation of [Hb]. Thus, a correct determination of [Hb] using Beer's Law requires that the light intensity distribution in the focal plane F be extracted.

Referring now in detail to the various figures of the drawing wherein like reference characters refer to like parts, there is shown at 20, a flow diagram of the method of the present invention. Thus, the following method 20 is implemented in the base unit 3 of the OPS imaging system. The method 20 is based on a physical model that relates the intensity distribution within the focal plane F to the quantity of light scattered in the region between the focal plane F (where vessels reside) and the tissue surface 12A.

The first step 22, requires that the polarized light 11 be emitted at the target tissue 12. In step 24, after the AOP 10 removes the surface-reflected light 13A, the de-polarized light 13B is passed to the base unit 3 where the measured light intensity I^{M}(x) forms an “original” or “subsampled” image. In step 26, the processor 4 estimates the intensity of the light scattered in the foreground tissue for every pixel 36, Iₛ(x). In step 28, the processor 4 subtracts the scattered light Iₛ(x) from the original image I^{M}(x) to determine light intensity in the focal plane F, I_{f}(x). In step 30, using this “corrected light intensity,” Beer's Law is then used to determine the concentration of hemoglobin.

In particular, in estimating the light scattered (Iₛ) in the foreground tissue 32 for every pixel 36 position, step 26 comprises:
a) Calculating the probability (Pₛ) that a photon originating from position xᵢ(xᵢ≠x) on the focal plane F scatters within foreground tissue 32 into the optical path 38 of the pixel 36 at x on the tissue surface 12. The probability (Pₛ) can be estimated using standard Monte Carlo computation methods.
b) Centering the probability function (Pₛ) on x and multiplying Pₛ with the image intensity distribution I^{M}. Summing over all xᵢ in the focal plane F to obtain the total intensity of scattered light Iₛ at x:

$${I}_{s} ( x ) = {\int }_{image} {( ⅆ {x}_{i} )}^{2} {P}_{s} ( \vert x - {x}_{i} \vert , {z}_{f} ) \cdot {I}^{M} ( {x}_{i} ) .$$

Step 28, subtracting scattered light Iₛ(x) from the original image I^{M}(x) to determine intensity in focal plane F, I_{f}(x), is given by:

$${I}_{f} ( x ) = \frac{{I}^{M} ( x ) - {I}_{s} ( x )}{exp ( - {μ}_{t} {z}_{f} )} .$$

Next, step 30, using Beer's Law to determine the concentration of hemoglobin [Hb]

$$[ Hb ] = - \frac{1}{α D} log ( \frac{{I}_{f} ( {x}_{v} )}{{I}_{f} ( {x}_{b} )} ) = - \frac{1}{α D} log ( \frac{{I}^{M} ( {x}_{v} ) - {I}_{s} ( {x}_{v} )}{{I}^{M} ( {x}_{b} ) - {I}_{s} ( {x}_{b} )} ) ,$$

where xᵥ and x_{b} are position of vessel and background, respectively; D is the diameter of the vessel BV and α is the absorptivity of Hb.

The parameters which contribute to the light scattered from other positions on the focal plane F (Iₛ(x)) are (1) depth z_{f} (parameter of probability function); (2) scattering coefficient (also a parameter of the probability function); and (3) light distribution in the focal plane F (e.g., shadows, diameter of main vessel).

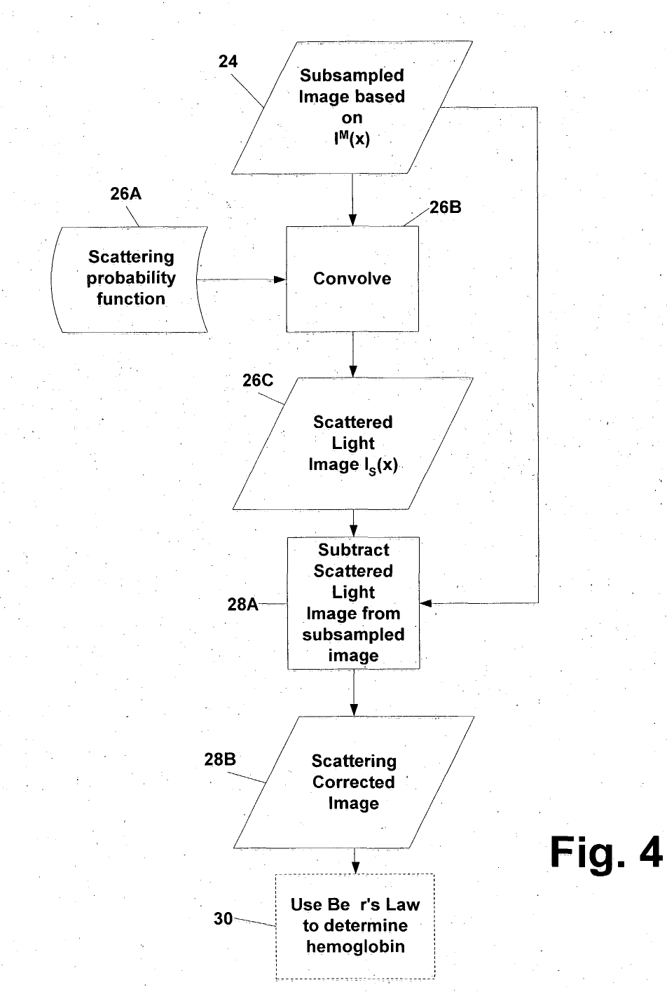

The method 20 of the present invention can be implemented in a computer program for use by the processor 4. As shown in the Appendix, by way of example only, computer code is written in MatLab language to perform the method 20 of the present invention. FIG. 4 provides an overview of the flow of the computer program. In particular, step 24 comprises generating the subsampled or original image from the measured light intensity, I^{M}(x). Step 26 comprises sub-steps 26A-26C, namely, calculating the scattering probability function (Pₛ) 26A, convolving the subsampled image and the probability function Pₛ 26B and generating the scattered light image, Iₛ(x) 26C. Next, step 28 comprises sub-steps 28A-28B: in step 28A, a scattered light image is subtracted from the subsampled image (forming step 28A) and a scattering corrected image based on I_{f}(x) is generated in step 28B. Although not shown in the computer program attached as Appendix, the processor 4 then calculates the concentration of hemoglobin [Hb] using Beer's Law, discussed earlier.

APPENDIX

```
function [ImgCorr, Ifore] = ForeScat (Img, simulate);
%%% Calculate the amount of light scattered within the foreground tissue.
    t0 = clock;
    Lpixel = 0.0002; %cm
    load ImgScat.mat;
    fprintf (‘Assumed focal depth = %.0f
microns/n’, z_focus*10\hat{ }4);
    mu_t = mu_a + mu_sp;
    %% Resize image so that pixel size is same as for ImgScat.
    ImgSize = size(Img);
    subsamp = dx_im/Lpixel;
    NewSize = ImgSize/subsamp;
    if ˜ (fix(NewSize) ==NewSize)
      fprintf (‘Sampling interval incorrect. \n’)
      return;
    end
    Img2 = imresize (Img, NewSize, ‘bilinear’);
    %% Determine intensity at focal plane.
    FocalImg = Img2/(exp(−mu_t*z_focus) + TotCylProb);
    if (‘simulate’ == simulate)
      FocalImg = Img2;
    end
    ImgScat = [ImgScat (:, end:−1:2) ImgScat];
    ImgScat = [ImgScat (end:−1:2,:); ImgScat];
    %% Determine amount of scattered light in foreground.
    Ifore = conv2 (FocalImg, ImgScat, ‘same’);
    Ifore = imresize (Ifore, ImgSize, ‘bilinear’);
    lmCorr = Img − Ifore;
    if (‘simulate’ == simulate)
      ImgCorr = Img*exp(−mu_t*z_focus) + Ifore;
    end
    fprintf (‘ Ig max = %.3f \n’, max (Ifore( : )) );
    fprintf (‘ Ig min = %.3f \n’, min (Ifore( : )) );
    fprintf (‘ Ig mean = %.3f \n’, mean (Ifore ( : )) );
    fprintf (‘ Time2 = %.1f\n’, etime (clock, t0) );
return;
function [rho, prob, CylProb] = ScatProbAll (z_focus, mu_a,
mu_sp, twoD );
%%% Calculate the probabilities of foreground scattering from
all pixels in
%%% focal plane. This function calls ScatProbl for all pixels.
%%% Robert Danen (5.21.99)
    t0 = clock;
%%  Definitions --------------------------------------------------
    Lpixel = 0.0002; %cm = size of pixel
    NA = 0.165; %numerical aperture of probe
    NA_theta = asin (NA);
    TanNA = tan (NA_theta );
    sNA = 2*pi* ( 1−cos(NA_theta) ); %solid angle of NA
%%  Setup sampling intervals --------------------------------
    if (twoD)
        ScatRegion = 0.024; %0.07; %cm
        subsamp = 2; %4;
    dx_im = subsamp*Lpixel; %sampling of final probability
matrix
        dx = dx_im/4;
        dx_2 = dx/2;
    else
        ScatRegion = 0.07; %0.128 %cm
        dx_im = 0.0006; %cm
        dx = dx_im;
    end
    rho = [0:dx:ScatRegion]; %rho = [0:dx:0.1278];
%%  Do calculations -------------------------------------------
    mu_t = mu_a + mu_sp;
    clear prob;
    prob(1) = 0;
    for i=2:length(rho) %DO NOT calculate for rho=0.
        rho1 = rho(i);
        prob(i) = ScatProb1 (rho1, z_focus, TanNA, Lpixel, mu_a,
mu_sp);
    %prob = ScatProbV(rho1, 0.0025, z_focus, 1.25*z_focus,
250, 57); prob(i) = sum(prob);
    end
    DetNormA = ( (dx/Lpixel)\hat{ }2) /sNA; %Normalize intensity
    DetNormB = ( (dx_im/Lpixed)\hat{ }2) /sNA; %Normalize intensity
%%------------------------------------------------------------------------------------
%%  IN-VITRO FACTOR
    prob = 5.0*prob;
% prob = 2.7*prob; %for g=0.8
%%------------------------------------------------------------------------------------
%%  Uniform (cylindrical) geometry %%
    CylProb = DetNormA*(2*pi/dx)*rho.*prob;
    TotCylProb = sum(CylProb);
    fprintf (‘ Scattering factor = %.4f \n’, TotCylProb );
%% Make 2D matrix containing scattering probabilities; save to
file.
    If(twoD)
        [X, Y] = meshgrid (0:dx_im:rho(end), 0:dx_im:rho(end));
        Z = sqrt (X.\hat{ }2+Y.\hat{ }2);
        [sy, sx] = size(Z);
        fprintf (‘ Quarter-size of probability image - %d x
%d\n’,sy,sx);
    ImgScat = zeros(sy,sx);
    for iy=1:sy
        rhol = Z (iy,ix);
        ind = find ( rho >=(rho1−dx_2) & rho < (rho1+dx_2) );
        if (˜isempty(ind))
            ImgScat (iy,ix) = prob(ind) *DetNormB;
        else
            ImgScat (iy,ix) = 0.0;
        end
    end
end
save ‘c:\RadiativeTransfer\ImgScat’ dx_im z_focus mu_a
mu_sp TotCylProb ImgScat;
    %save ‘c:\RadiativeTransfer\ImgScat.m’ ImgScat -ASCII;
end
fprintf(‘ Time1 = %.1f\n’, etime(clock,t0) );
function prob = ScatProb1(rho1, z_focus, TanNA, Lpixel, mu_a,
mu_sp)
%% Calculates probability that a photon originating at a
distance
%% rho1 in the focal plane from the pixel of interest scatters
into
%% the collection cone of the pixel.
%% Robert Danen (5.21.99)
    t0 = clock;
    g = 0.8;
%%  Definitions-----------------------------------------------------------------
    mu_t = mu_a + mu_sp;
    Dtheta2 = 0.01; %radians
    Dphi3 = 0.01; %radians
    Drho3 = 0.00002; %cm
%%  Do calculations-----------------------------------------------------------
    %Theta2_lim = atan(z_focus/rho1);
    Theta2_lim = abs ( atan(1.0/( (rho1/z_focus) − TanNA) ) );
    Theta2 = [Dtheta2:Dtheta2:Theta2_lim];
    CosTheta2 = cos(Theta2); %cos theta
    SinTheta2 = sin(Theta2);
    r2 = rho1./CosTheta2;
    SA_source = 2*sin(0.5*Dtheta2)*Dphi3;
    source = SinTheta2.*SA_source;
    clear Zprob;
    for ith2 = 1:length(Theta2)
        %fprintf(‘ith2= %d th2=
%.3f\n’ith2,acos(CosTheta2(ith2) ) );
    phi3_lim_a = atan( SinTheta2(ith2) * TanNA );
    phi3_lim = fix(phi3_lim_a/Dphi3)*Dphi3;
    if (phi3_lim == phi3_lim_a)
      phi3_1im = phi3_lim − 1;
    end
    phi3 = pi + [0:Dphi3:phi3_lim];
    %% determine length where scattering event can occur
(from rho3_lim1 to rho3_lim2)
    [rho3_lim1, rho3_lim2] = ConeIntersection(phi3,
CosTheta2 (ith2), SinTheta2 (ith2), rho1, TanNA);
    r1_lim2 = CoordinateTrans (rho3_lim2, phi3,
CosTheta2 (ith2), SinTheta2(ith2), rho1 );
    ind = find(r1_lim2 (:,3) > z_focus | r1_lim2 (:,3) < 0);
    rho3_lim2 (ind) = −z_focus./(
SinTheta2(ith2)*cos(phi3 (ind) ) );
    %%% Integrate over rho3 for each phi3. %%%
    clear Zprob1;
    for iphi3 = [1:length(phi3)]
        clear rho3;
        rho3 = [rho3_lim1 (iphi3):Drho3:rho3_lim2(iphi3) ];
        %clear p1 p2 p3;
        p1 = exp (−mu_t*rho3);
        r1_s = CoordinateTrans(rho3, phi3(iphi3),
CosTheta2(ith2), SinTheta2 (ith2), rho1);
    %r1_out = z_focus −r1_s(:,3);
    r1_s_M2 = sum(r1_s.\hat{ }2,2);
    r1_s_M2 = r1_s_M2′;
    r1_s_M = sqrt( r1_s_M2 );
    SecTheta1 = r1_s_M./ (r1_s(:,3) ′ );
    r1_out = (z_focus − r1_s(:,3) ′).*SecTheta1;
    p3 = exp(−mu_t*r1_out);
    dtheta1 = 0.5*Lpixel./(r1_s(:,3) ′ );
    ind = find(dtheta1 > TanNA);
    dtheta1(ind) = TanNA;
    dtheta1 = atan( dtheta1 );
    SApixel = 4*dtheta1.*sin(dtheta1); %=
4*dtheta1.*sin(dtheta1)
    %theta_s = acos( (rho3.\hat{ }2 + r1_s_M2 −
rho1\hat{ }2)./(2*r1_s_m.*rho3) );
    %ptheta = SApixel.*HGPhase(theta_s,g);
    ptheta = Sapixel/(4*pi); % (for mu_s′) % =)(solid
angle of pixel)/4pi
    Zprob1(iphi3) = sum( ptheta.*p1.*p3 );
    end
    Zprob(ith2) = 2*sum(Zprob1) − Zprob1(1); %Integrate over
only half the NA cone to save computational time.
    end
    Zprob = source.*Zprob;
    p2 = 1 − exp(−mu_sp*Drho3);
    prob = p2 * sum( Zprob ); %multiply by 2 because only
integrated over half of cone
%  fprintf(‘elapsed time = %.1f\n’, etime(clock,t0) );
%  fprintf(‘prob = %.5e \n’, prob);
return;
%%%%%%%%%%%%%%%%%%%%%%%%%%%%%%%%%%%%
%%
function [rho3_a, rho3_b] = ConeIntersection(phi3, CosTheta2,
SinTheta2, xshift1, TanNA)
%%  Calculate roots of cone in cylindrical frame
%%  phi3 is a vector, remaining inputs must be scalar.
```

| | |
|---|---|
| CosPhi3 | = cos (phi3); |
| SinPhi3 | = sin (phi3); |
| CosPhi3_2 | = CosPhi3.\hat{ }2; |
| SinPhi3_2 | = SinPhi3.\hat{ }2; |
| CosTheta2_2 | = CosTheta2\hat{ }2; |
| SinTheta2_2 | = SinTheta2\hat{ }2; |
| TanNA_2 | = TanNA\hat{ }2; |

| | |
|---|---|
| term1 | = (SinTheta2_2 * TanNA_2) * CosPhi3_2); |
| denom | = (term1 − CosTheta2_2*CosPhi3_2 − SinPhi3_2 ); |
| term2 | = sqrt ( term1 − SinPhi3_2 ); |
| term3 | = CosTheta2*CosPhi3; |
| term4 | = xshift1./denom; |
| rho3_a | = term4.*(term3 + term2); |
| rho3_b | = term4.*(term3 − term2); |

```
return;
%%%%%%%%%%%%%%%%%%%%%%%%%%%%%%%%%%%%
%%
function r1 = CoordinateTrans (rho3, phi3, CosTheta2,
SinTheta2, xshift1)
%% Transform from source to detection coordinate frames, for
z3=0.
%% rho and phi3 are vectors with same index, remaining inputs
must be scalar.
    CosPhi3 = cos(phi3);
    SinPhi3 = sin(phi3);
    r1(1,:) = CosTheta2*(rho3.*CosPhi3) + xshift1;
    r1(2,:) = rho3.*SinPhi3;
    r1(3,:) = −SinTheta2*(rho3.*CosPhi3);
    r1 = r1′:
return;
function prob = HGPase (theta,g);
%%% Normalized Henyey-Greenstein phase function.
    a = 1 − g\hat{ }2;
    b = 2*(1 + g\hat{ }2 − 2*g*cos(theta)).\hat{ }1.5;
    prob = a./(2*pi*b);
return;
```

1. A method for correcting light intensities from blood vessels and background tissue beneath tissue surface in a living being that are exposed to polarized light from an orthogonal polarized spectral (OPS) imaging system, said method comprising the steps of:
emitting polarized light at tissue comprising blood vessels and background tissue, the blood vessels being located at a focal plane of said OPS imaging system and wherein a foreground region is formed between said focal plane and the tissue surface;
collecting de-polarized light that has impacted the blood vessels and that has experienced scattering within said foreground region, said de-polarized light emerging from the tissue surface;
generating a first image based on said collected de-polarized light;
estimating the intensity of light scattered in the foreground region; and
subtracting said intensity of light scattered in the foreground region from said first image to generate a corrected image based on focal plane light intensities.

2. The method of claim 1 wherein said method of estimating-the intensity of light scattered in the foreground region comprises:
generating a probability function based on a photon of said de-polarized light originating from said focal plane and scattering within said foreground region;
centering said probability function along an axis within said focal plane and multiplying said probability function with said first image to form a light intensity of scattered light; and
summing all of said scattered light intensities from said focal plane;

3. The method of claim 2 further comprising the step of inputting said scattered light intensities, said focal plane light intensities, the absorptivity of hemoglobin and the diameter of said blood vessels into Beer's Law to determine hemoglobin concentration.

4. The method of claim 2 wherein said steps of centering said probability function along an axis within said focal plane and multiplying said probability function with said first image to form a light intensity of scattered light and summing all of said scattered light intensities from said focal plane comprises utilizing the following relationship;
$${I}_{s} ( x ) = {\int }_{image} {( ⅆ {x}_{i} )}^{2} {P}_{s} ( \vert x - {x}_{i} \vert , {z}_{f} ) \cdot {I}^{M} ( {x}_{i} )$$
wherein
x=an axis within the focal plane;
Iₛ(x)=the light intensity of scattered light;
Pₛ=probability function based on a photon of said de-polarized light originating from the focal plane at a location xᵢ along said x axis; and
I^{M}(x)=said collected de-polarized light that has impacted the blood vessels and that has experienced scattering within said foreground region and emerged from the tissue surface.

5. The method of claim 4 wherein said step of subtracting said scattered light intensities from said first image to determine focal plane light intensities comprises utilizing the following relationship:
$${I}_{f} ( x ) = \frac{{I}^{M} ( x ) - {I}_{s} ( x )}{exp ( - {μ}_{t} {z}_{f} )}$$
wherein,
I_{f}(x)=focal plane light intensities;
μₜ=is the transport coefficient of the foreground region; and
Z_{f}=the distance between said foreground region and the tissue surface.

6. The method of claim 5 further comprising the determining the hemoglobin concentration in the blood vessel using the following relationship:
$$[ Hb ] = - \frac{1}{α D} log ( \frac{{I}_{f} ( {x}_{v} )}{{I}_{f} ( {x}_{b} )} ) = - \frac{1}{α D} log ( \frac{{I}^{M} ( {x}_{v} ) - {I}_{s} ( {x}_{v} )}{{I}^{M} ( {x}_{b} ) - {I}_{s} ( {x}_{b} )} )$$
wherein,
[Hb]=concentration of hemoglobin;
α=absorptivity of hemoglobin;
D=diameter of the blood vessel
I_{f}(xᵥ)=focal plane light intensity evaluated at vessel x-axis position;
I_{f}(x_{b})=focal plane light intensities evaluated at background x-axis position;
Iₛ(xᵥ)=the light intensity of scattered light evaluated at vessel x-axis position;
Iₛ(x_{b})=the light intensity of scattered light evaluated at background x-axis position;
I^{M}(xᵥ)=said collected de-polarized light that has impacted the blood vessels and that has experienced scattering within said foreground region and emerged from the tissue surface evaluated at vessel x-axis position; and
I^{M}(x_{b})=said collected de-polarized light that has impacted the blood vessels and that has experienced scattering within said foreground region and emerged from the tissue surface evaluated at background x-axis position.
